(19) United States
(12) Patent Application Publication
(10) Pub. No.: US 2003/0130624 A1
(43) Pub. Date: Jul. 10, 2003

(54) Medical infusion system with integrated power supply and pump therefor

(76) Inventors: Francis C. Kowalik, Deerfield, IL (US); James D. Jacobson, Lindenhurst, IL (US)

Correspondence Address: Wallenstein & Wagner, Ltd., 53rd Floor, 311 S. Wacker Drive, Chicago, IL, 60606-6622, US

(21) Appl. No.: 10/040,908

(22) Filed: Jan. 7, 2002

(51) Int. Cl. A61M 5/00

(52) U.S. Cl. 604/246; 604/67

(57) ABSTRACT

A medical infusion system (10) including a lineset used for delivering fluid, such as a liquid medicinal substance, to a patient from a source such as an IV bag through operation of an electric component is disclosed. The preferred infusion system includes disposable tubing having first and second ends attachable to at least a first and second medical component, and a power supply, such as a fuel cell, battery, battery pack, power paper, or a combination of the same, attached to the tubing wherein the power supply is configured to be activated to provide electric power to the electric component. Such configuration may include the use of an activating member, such as a fluid pump. The preferred power supply is a fuel cell having a reactant source and a barrier separating the reactant source from a reaction chamber. The barrier is preferably selected from the group consisting of a frangible membrane, a tear seal, and any combination of the two. Additionally, the fuel cell (or the power supply, generally) may be integral to the tubing of the lineset or may be configured to fit within the fluid pump. This allows the activating member to be made integral to the fluid pump such that the insertion of the fuel cell into the pump will defeat the barrier and activate the fuel cell to create power.

### DESCRIPTION

1. Technical Field

The present invention relates to powering delivery of fluid from a fluid source to, for example, a patient. Specifically, the present invention relates to an economical and ecologically friendly source for powering a fluid pump, particularly a portable fluid pump.

2. Background of the Invention

Generally, medical patients require precise delivery of either continuous medication or medication at set periodic intervals. Medical pumps have been developed to provide controlled drug infusion through the pump wherein the drug can be administered at a precise rate that keeps the drug concentration within the therapeutic margin and out of a possible toxic range with certain drugs. The medical pumps provide appropriate drug delivery to the patient at a controllable rate which does not require frequent medical attention. The medical pumps further facilitate administration of intravenous therapy to patients outside of a clinical setting. In addition, doctors have found that in many instances patients can return to substantially normal lives, provided that they can receive periodic or continuous intravenous administration of medication. Among the types of therapies requiring this kind of administration are antibiotic therapy, chemotherapy, pain control therapy, nutritional therapy, and several other types known by those skilled in the art. In many cases, patients may receive multiple daily therapies. Certain medical conditions require infusions of drugs in solution over relatively short periods such as from 30 minutes to two hours. These factors have combined to promote the development of increasingly lightweight, portable or ambulatory infusion pumps that can be worn by a patient and are capable of administering a continuous supply of medication at a desired rate, or several doses of medication at scheduled intervals.

The different types of infusion pumps in the prior art include elastomeric pumps which squeeze the solution from flexible containers, such as balloons, into IV tubing for delivery to the patient. Elastomeric pumps require no electric power, have no programming capabilities, and have relatively poor accuracy compared to electromechanical pumps.

Spring-loaded pumps have also been provided to pressurize the solution containers or reservoirs. Certain pump designs utilize cartridges containing flexible compartments that are squeezed by pressure rollers for discharging the solutions, such as in U.S. Pat. No. 4,741,736. Other references which disclose portable infusion pumps include U.S. Pat. No. 5,330,431 (showing an infusion pump in which standard pre-filled single dosage IV bags are squeezed by the use of a roller); U.S. Pat. No. 5,348,539 (showing an infusion pump in which prepackaged IV bags are squeezed by a bladder which is actuated by fluid pumped from a reservoir); U.S. Pat. No. 5,429,602 (showing a programmable portable infusion pump system for injecting one or more medicinal substances into an individual); and U.S. Pat. No. 5,554,123 (showing an infusion pump in which fluid is moved from a reservoir by a peristaltic pump into a pressure chamber). Typically, these ambulatory infusion pumps include a pump control unit, a drive mechanism including a variety of operating controls adapted to accept a disposable pump chamber assembly, and a power source for powering the pump and controls. In most cases, the pump chamber assembly has an inlet end connected to a liquid reservoir, such as an I.V. bag, and an outlet end connected to an I.V. tube that in turn is connected for intravenous administration to a patient by an access device such as a needle, catheter, cannula, or the like.

While the discussed prior art and other designs have recognized the need for an infusion pump which is smaller and more compact for mobile use by ambulatory and other patients, each has failed to address the need for a more suitable power source. Naturally, a portable pump must be supplied with an equally portable power source as a means for powering the pump motor. In prior art pumps, large cell batteries or battery packs within the pumps have typically been used to provided the necessary power. Some problems may exist with the use of larger and heavier battery sizes (9 volt, “D”, and “C” sizes, for example), but an embodiment of the present invention could be conceived to incorporate such design parameters.

One specific example of prior art recognizing these problems is illustrated by the International Application PCT/US84/00526, published on Feb. 14, 1985 under Publication No. WO 85/00523. This reference teaches the attachment of a battery to a flexible, collapsible solution container which is used to operate the pump. This innovative solution, however, is limited to use with the specific pump type allowing insertion of the solution container. The present invention has broader applications.

In other devices the batteries and battery packs may be large and bulky, adding significantly to the weight of the portable pump. Weight and size of the infusion pump is an important consideration because it may be carried about by patients attempting to maintain their rigorous daily schedules. Where interrupted operation of the pump may have negative consequences, extra batteries or an extra battery pack may be added to the carrying necessities of the infusion pump. In some instances the carrying of a second set of batteries or a back-up battery pack may double the weight of the power source.

Additionally, where such batteries or battery packs are rechargeable, an AC outlet is usually necessary. A separate charger, as is well-known in the art, is also usually required for the recharging effort. Unfortunately, these facilities are not always readily available or accessible to the patient and, with respect to the usual adapters and extension cords, they will add to the bulk and weight of the infusion pump system.

Finally, where the batteries are not rechargeable, there is an environmental disposal concern, as these little energy supplies place a considerable burden on the environment. Non-rechargeable batteries are responsible for a major share of heavy metal pollution in domestic waste. Despite special collection efforts and consumer awareness campaigns, a high percentage of batteries sold still end up in domestic waste sites. Here the heavy metals they contain eventually leak into the ground soil and lead to damage of the environment, with a greater potential for adverse affects to human health.

The present invention provides a portable, preferably disposable power source for use with a durable, portable pump which solves these and other problems either ignored by prior art designs or unappreciated by those skilled in the art.

### SUMMARY OF THE INVENTION

The present invention provides a medical infusion system used for delivering fluid, such as a liquid medicinal substance, to a patient from a source such as an IV bag through operation of an electromechanical component. The lineset includes tubing having first and second ends attachable to at least a first and second medical component, and a power supply attached to other than the electric component (e.g., the tubing) and configured to be activated to provide electric power to the electric component by use of an activating member.

In one embodiment of the present invention the electromechanical component is a fluid pump. It may be any of the types of fluid pumps known by those skilled in the art, including programmable, portable, and multichannel pumps.

It is an aspect of the invention to provide, as the power supply, a fuel cell having a reactant source and a barrier separating the reactant source from a reaction chamber. The barrier is preferably selected from the group consisting of a frangible membrane, a tear seal, and any combination of the two.

In another embodiment of the invention, the power supply is made integral to the tubing of the lineset. It is further an aspect of this embodiment to configure the power supply to fit within the fluid pump. This requires an activating member to be made integral to the fluid pump, such that by the insertion of the power supply into the pump the barrier will be defeated and the power supply will be activated to create power.

The present invention also provides a method for powering a fluid pump with a separate power supply. The preferred method includes the steps of providing tubing with an attached power supply, such as a fuel cell, operably connecting the power supply to the fluid pump, and then activating the power supply to provide electrical power to the fluid pump. The fuel cell, for example, operates by providing a suitable reactant to a reaction chamber of the fuel cell to cause a chemical reaction. By defeating a barrier separating the reactant from the reaction chamber within the fuel cell the reaction is allowed to take place.

The barrier may be defeated or overcome by any number of methods, including removing a tear seal or breaking a frangible membrane, or any combination of the two. The method preferably includes the step of operably connecting the fuel cell to a fluid pump by placing the fuel cell into a compartment of the fluid pump.

The present invention also includes a method for delivering fluid through a lineset which includes providing tubing having a first end in fluid communication with a fluid source and a second end in fluid communication with a delivery device, providing a power supply operably connected to a fluid pump, activating the power supply to provide power to the fluid pump, and pumping fluid through the tubing from the fluid source toward the second end of the tubing.

These and other advantages are provided by the invention of the present application as described in the following specification and appended drawings.

### BRIEF DESCRIPTION OF THE DRAWINGS

To understand the present invention, it will now be described by way of example, with reference to the accompanying drawings in which:

FIG. 1 is a schematic illustrating one embodiment of the present medical infusion system having an integrated power supply affixed to a lineset component, such as a valve or sensor, and alternatively, affixed directly to the medical tubing;

FIG. 2 is a schematic illustrating the embodiment of FIG. 1 as the power supply might operably connect to a pump;

FIG. 3 is a schematic illustrating the operable connection of the embodiment of FIG. 2;

FIG. 4 is a schematic illustrating the operable connection of an alternative embodiment (i.e., the embodiment of FIG. 1 shown in broken lines) of the present invention;

FIG. 5 is a schematic illustrating the use of a fuel cell to recharge the power supply for powering the pump; and

FIG. 6 is a schematic showing, generally, the components of a PEM fuel cell power supply, and illustrating two possible placements for an activating member.

### DETAILED DESCRIPTION

While the present invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

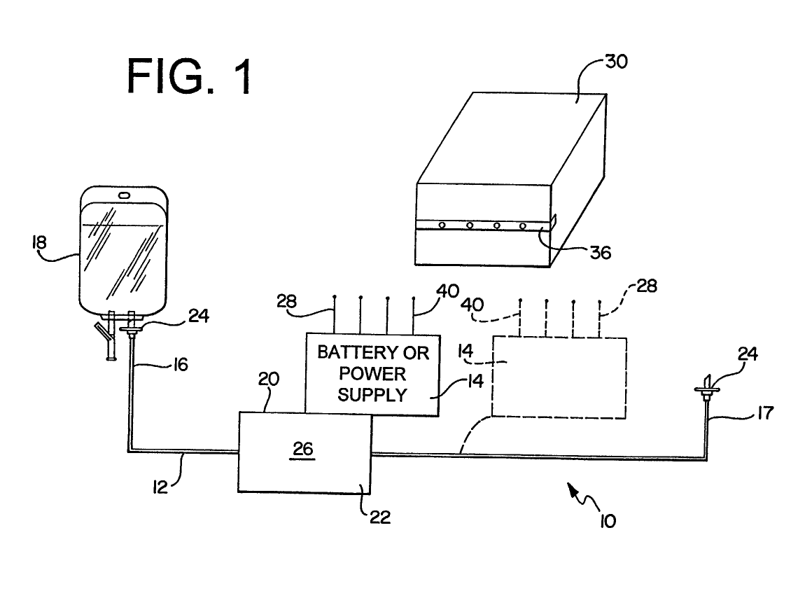

Referring generally to the appended FIGS. 1-6, the apparatus and method for delivering fluid from a fluid source to a patient using the present invention can be more readily understood. The disclosed infusion system is generally referenced by the number “10” in the following disclosure and drawings. Other components are similarly and consistently numbered throughout the specification and drawings. While the present invention is particularly designed for use with a portable infusion pump, other such fluid pumps and electric medical devices may be capable of adaptation for implementation of the system as well. Such pumps requiring modification may include, for example, the COLLEAGUE® Volumetric Infusion Pump, the FLO-GARD® Volumetric Infusion Pump, the AUTO SYRINGE® Infusion Pump, or the maxx® Infusion System, and their progeny, designed and manufactured by Baxter International, Inc. of Deerfield, Ill.

As shown in FIG. 1, the present system 10 is generally comprised of a section of tubing 12 having a first end 16 and a second end 17, and, in one embodiment, an attached power supply 14 between the two ends. The first end 16 of the tubing 12 is shown configured for connection, for example, to a fluid source such as an IV bag 18, while the second end 17 of the tubing 12 is configured for connection to, for example, an injection port (not shown). The power supply 14 is preferably attached to an outer surface 20 of a lineset component 22, such as a valve, flow sensor, pump, pressure sensor, feedback control input, biological status sensor, or other closed loop sensor know to those skilled in the art. However, as shown by the broken lines of FIG. 1, the power supply 14 may be affixed directly to the tubing 12 at any point between the tubing ends, 16 and 17, respectively.

The tubing 12 can be of any suitable medical grade tubing used for procedures requiring a transfer of fluid from at least one source site to at least one recipient site. Exemplary tubing is described in U.S. patent application Ser. No. 08/642,278, entitled “Method of Using Medical Tubings in Fluid Administration Sets,” and U.S. Pat. No. 6,129,876, entitled “Heat Setting of Medical Tubing,” each filed on May 3, 1996, and assigned to the Assignee of this application. Each of these documents is hereby incorporated by reference.

The tubing 12 has a first end 16 which, in a preferred embodiment, has a connector 24, such as a spike connector, for attachment of the tubing 12 to a fluid source (a first component) such as, for example, an IV bag 18. A second end 17 of the preferred tubing 12 can be equipped with a connector 24 for attachment to, for example, a cannula, catheter, syringe, IV line, or any of several other known medical instruments or devices (a second component).

While the system 10 of FIG. 1 shows a single line system, it is within the scope of the present invention to encompass multiple fluid lines. Such a configuration may be necessary where, for example, more than one medical substance is to be injected into a patient.

The system 10, as shown in FIG. 1, is also comprised of a uniquely configured power supply 14. The power supply 14 may be attached directly to the tubing surface via connector 34 (dashed power supply 14) or indirectly to the tubing surface, or it may be attached to another component of the system 10. The power supply 14 may come in a variety of forms, including various battery sizes (e.g., D, C, AA, AAA, or 9 volt sizes), but is preferably a fuel cell, or alternatively a flexible thin layer open electrochemical cell, the latter of which is discussed in U.S. Pat. No. 5,897,522 and hereby incorporated by reference.

As a further alternative, the power supply 14 may be a means for inputting AC power to the pump component. This may include an inductor attached to the lineset, or any other acceptable means known by those skilled in the art. The use of an additional battery, such as a coin cell (or button) battery, is contemplated for inclusion in the durable pump component of the present invention. This power source (not shown) could be used to run and maintain memory functions of the pump or durable component.

A suitable casing 26 to house the power source may be desirable for some applications. In such a case, suitable connectors, such as electric leads 28, for example, may be used to operable connect the power supply 14 (i.e., the encased power source) to the durable pump 30. In light of this teaching, providing such a housing and connectors would be readily understood by those skilled in the art.

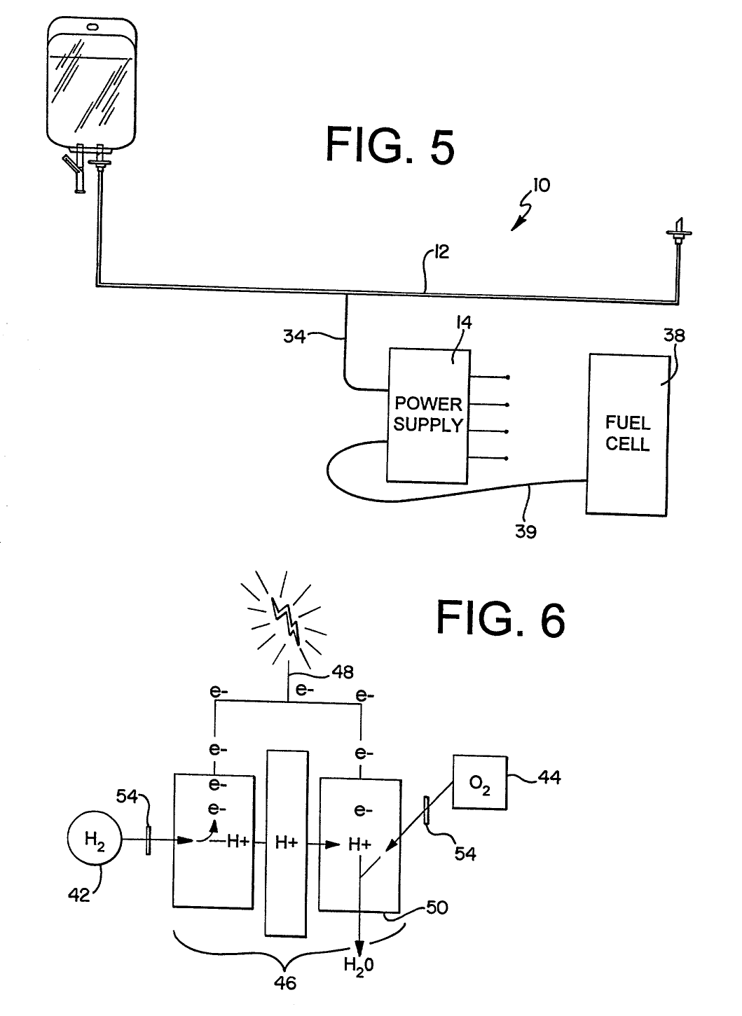

In still other alternative embodiments, as illustrated in FIG. 5, the use of a fuel cell 32 may be to either power the pump 30, as described above, or to recharge a power supply 14 (such as rechargeable batteries) that in turn powers the pump 30. The recharging fuel cell 38 may be a separate component that operably connects to the power supply 14, or it may be affixed or integral to the durable pump 30. The recharging fuel cell 38 could be connected to the power supply 14 via electrical connector 39 either continuously, periodically, or as needed (i.e., when the energy of the power supply 14 reaches a minimum threshold level). Activation of the recharging fuel cell 38 may be by conventional methods known to those skilled in the art, or in the manner described below.

In one embodiment utilizing a fuel cell, the fuel cell 32 is provided as an integral component to an outer surface of the tubing 14. By “integral” it is meant that the fuel cell 32 is permanently attached to the tubing surface by any suitable means. While the present drawings and description refer to a polymer electrolyte membrane fuel cell (PEM-FC), other types of fuel cells may be suitable, preferably low-temperature fuel cells. However, such other types including phosphoric acid, solid oxide, alkaline, direct methanol, and regenerative type fuel cells may be acceptable. Permanent attachment of the power source to the tubing provides certainty regarding power availability and life. That is, by making the power supply 14 part of the disposable component of the infusion system 10 a healthcare practitioner would not need to track the usage of the durable pump batteries, stock batteries of various sizes, or change batteries during an infusion regimen.

The fuel cell 32 may be any of the myriad of fuel cell designs available and suitable for such use. Exemplary fuel cell designs are disclosed in U.S. Pat. No. 5,976,725, entitled “Fuel Cell System, Fuel Feed System For Fuel Cell And Portable Electric Appliance” and issued Nov. 2, 1999 and U.S. Pat. No. 5,723,229, entitled “Portable Fuel Cell Device Including A Water Trap” and issued Mar. 3, 1998.

As an alternative power source to the fuel cell, flexible thin layer open electrochemical cells may be used. These “batteries” (a.k.a. “Power Paper”) are described in U.S. Pat. No. 5,897,522 issued Apr. 27, 1999, to Nitzan and assigned to Power Paper Ltd., of Kibbutz Einat, Israel. Power Paper can be printed, pasted, or laminated onto paper, plastic, and other media. It can be made in almost any shape and size, while remaining flexible, inexpensive, safe, non-toxic, and simple to produce.

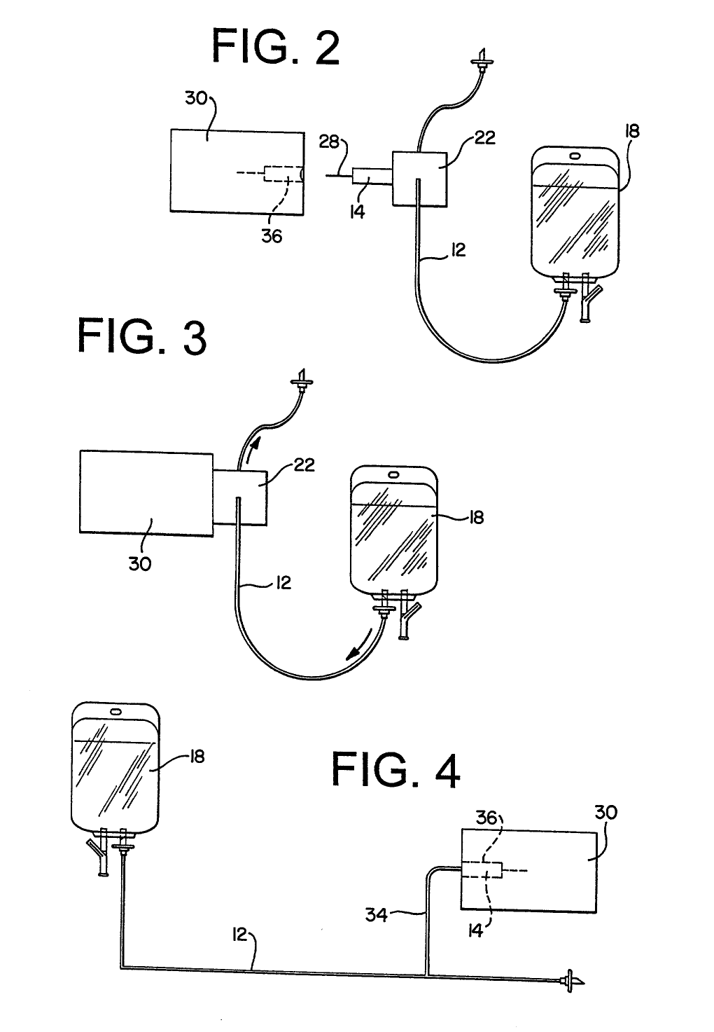

Referring to FIGS. 2 and 3, a power supply 14 is shown being inserted into a power supply compartment 36 of a fluid pump device 38. Where the power supply 14 is attached to a separate lineset component 22 of the lineset, the attachment may be operable for the component 22. That is, the power supply connection, via electric leads 28, for example, may activate the pump 30 as well as providing a link between the durable pump 30 and the component 22. It is possible additional contact may be necessary between the pump 30 and the tubing 12 to effect fluid flow. Those skilled in the art would understand the manner in which such connection may be made.

Alternatively, the power supply 14 may connect to the tubing 12—or any component other than the pump 30—via a connector 34, as shown in FIG. 4. In such a case, the tubing 12 may need to be placed within the pump 30 itself to permit pumping of fluid.

Referring again to FIG. 1, with respect to the use of a fuel cell, the pump power supply compartment 36 may comprise (as a component of the fluid pump 30) an activating mechanism or member 40 which activates the fuel cell 32 to begin production of electric power. The activating member 40—shown as a component of the power supply 14, but the reversal of the male and female components are contemplated—is preferably comprised of at least one electric contact linked to the pump motor (not shown) and capable of operably connecting to the fuel cell 32. Electric leads 28 are but one of a myriad of electric contact designs which may be suitable to provide activation of the fuel cell (or power supply, generally) when linked together as shown. Those skilled in the art would be cognizant of such alternatives, and the use of such alternatives should not be considered to be outside the scope of protection afforded the present application.

As illustrated in FIG. 6, a preferred low temperature fuel cell 32 generally includes a fuel (H₂) reservoir 42, an oxidant (O₂) reservoir 44, including respective feed-lines which couple to a reaction chamber 46, electric contacts 48 (see FIG. 1), and an exhaust line 50. In operation, generally, a fuel and an oxidant are delivered through feed-lines of the respective reservoirs, 42 and 44, to the reaction chamber 46 to combine and form a reactant mixture. Within the reaction chamber the fuel-oxidant (reactant) mixture is allowed to react in a known manner to produce electricity. The resulting electricity is transferred, for example, through the contacts 48 to the pump 30. Exhaust gases can be discharged to the environment or another device through the exhaust line 50.

A feature of a preferred fuel cell design is also illustrated in FIG. 6. A barrier 54 is utilized to prevent the requisite electricity-generating chemical reaction. There are a variety of ways to maintain separation between the reactants (i.e., the fuel and oxidant) and the reaction chamber 46. The barrier 54 may prevent fuel reactant flow (as with barrier 54a), oxidant reactant flow (as with barrier 54b), or the barrier may be set up in some other manner with the general intent of preventing electricity generation while the lineset 10 is not operably connected to the fluid pump 30.

One possible barrier design is a tear seal (not shown), as known by those skilled in the relevant art. The tear seal can be designed for removal—also referred to as defeating the barrier—by hand either before insertion of the fuel cell 32 into the fluid pump 30, or after the fuel cell 32 has been set into position. After removal of the tear seal barrier and insertion into the fluid pump 30, the contacts 48 engage the activating member 40 of the fluid pump 30.

Similarly, a frangible membrane may provide the necessary barrier 54. The membrane can also be designed for defeat before or after insertion into the power supply compartment 36. The activating member 40 may provide the barrier defeating device as well as the operable connection for the fuel cell 32 to the pump 30 through the contacts 48. The draw of electricity from the contacts 48 of the fuel cell 32 is typically used to drive the flow of reactants to the reaction chamber 46. That is, the fuel cell 32 operates on a demand basis.

With respect to the fluid pump 30, the present invention may utilize any of several known pump designs. While portable infusion pumps may be particularly suitable for the present technology advancement, larger, non-portable pumps may also realize particular advantages. For example, the use of the fuel cell 32 is environmentally friendly. Resulting exhaust gases are mostly harmless as opposed to the heavy metals of many dry cell batteries. The preferred fuel cells contain no heavy metals to cause environmental concern.

Additionally, the fuel reservoir 42 and oxidant reservoir 44 of the fuel cell 32 may be easily and quickly replenished. This provides a considerable advantage over prior art batteries used to presently power, for example, portable infusion pump devices.

The method of powering the fluid pump 30 with the fuel cell 32 begins by providing tubing 12 with an attached fuel cell 32, as illustrated in FIG. 1. Then, operably connecting the fuel cell 32 to the fluid pump_30 to activate the fuel cell 32. Connection is preferably achieved by inserting the fuel cell 32 within a compartment 36 of the pump 30. At this point the fuel cell 32 should begin to provide electrical power to the fluid pump 30.

The step of activating the fuel cell includes providing a suitable reactant to the fuel cell reaction chamber 46 to cause a chemical reaction. In a later step, it is necessary to defeat the barrier 54 separating the reactant mixture from the reaction chamber 46 within the fuel cell 32. As previously discussed, the barrier defeating step can be accomplished to activate the fuel cell 32 in many numerous ways, including removing a tear seal, breaking a frangible membrane, or any combination of the two.

Finally, as power is provided to the pump 30 fluid can be pumped through the tubing 12 from a fluid source such as IV bag 18 toward the second end 17 of the tubing 12, as illustrated in FIG. 3.

While the specific embodiments have been illustrated and described, numerous modifications can be made to the present invention, as described, by those of ordinary skill in the art without significantly departing from the spirit of the invention. The breadth of protection afforded this invention should be considered to be limited only by the scope of the accompanying claims.

We claim:

1. A medical infusion system comprising a lineset having a first end capable of attachment to a reservoir and a second end capable of attachment to another component, a durable pump component for engaging the lineset and controlling a fluid flow through the lineset, and a power supply affixed to other than the durable pump component and capable of operative connection with the durable pump component.

2. The medical infusion system of claim 1, wherein the power supply is affixed to the lineset.

3. The medical infusion system of claim 2, wherein the lineset and power supply are disposable.

4. The medical infusion system of claim 1, further comprising an auxiliary component attached to the lineset selected from the group consisting of a valve, a flow sensor, a pump, a pressure sensor, a feedback control input, a biological status sensor, other closed-loop type sensors, and any combination of such components.

5. The medical infusion system of claim 4, wherein the power supply is affixed to the auxiliary component.

6. The medical infusion system of claim 1, wherein the power supply comprises a fuel cell.

7. The medical infusion system of claim 1, wherein the power supply comprises means suitable for input of AC power.

8. The medical infusion system of claim 1, wherein the power supply comprises a battery.

9. The medical infusion system of claim 8, wherein the battery comprises a flexible thin layer open electrochemical cell.

10. The medical infusion system of claim 1, wherein the power supply is configured to be activated to provide electric power by an activating member.

11. The medical infusion system of claim 10, wherein the activating member is a component of the durable pump which operably connects to the power supply.

12. The medical infusion system of claim 6, wherein the fuel cell comprises a reactant source and a barrier separating the reactant source from a reaction chamber.

13. The medical infusion system of claim 12, wherein the barrier is selected from the group consisting of a frangible membrane, a tear seal, and any combination of the two.

14. The medical infusion system of claim 6, wherein the fuel cell is a low temperature fuel cell.

15. The medical infusion system of claim 1, wherein the power supply is integral to a surface of the lineset.

16. The medical infusion system of claim 1, wherein the power supply is configured to fit within the durable pump component.

17. The medical infusion system of claim 1, further comprising a recharger for recharging the power supply.

18. The medical infusion system of claim 17, wherein the recharger comprises a fuel cell.

19. A medical lineset comprising:
tubing having first and second ends attachable to at least a first and second medical component;
a power supply attached to the tubing; and
an activating member for placing the power supply into operative connection with an electric component.

20. The medical lineset of claim 19, wherein the activating member is a connector of the electric component.

21. The medical lineset of claim 19, wherein the power supply comprises a fuel cell.

22. The medical lineset of claim 21, wherein the fuel cell comprises a reactant source and a barrier separating the reactant source from a reaction chamber.

23. The medical lineset of claim 22, wherein the barrier is selected from the group consisting of a frangible membrane, a tear seal, and any combination of the two.

24. The medical lineset of claim 21, wherein the fuel cell is a low temperature fuel cell.

25. The medical lineset of claim 19, wherein the power supply is integral to a surface of the tubing.

26. The medical lineset of claim 25, wherein the power supply comprises a low temperature fuel cell.

27. The medical lineset of claim 20, wherein the fuel cell is configured to fit within the electric component.

28. The medical lineset of claim 22, wherein the barrier is configured to be defeated by a mechanism of the electric component.

29. The medical lineset of claim 27, wherein the fuel cell comprises a reactant source and a barrier separating the reactant source from a reaction chamber, the barrier being configured to be defeated by a mechanism within the fluid pump.

30. A method of powering a fluid pump comprising the steps of:
providing tubing with an attached power supply;
operably connecting the power supply to the fluid pump; and
activating the power supply to provide electrical power to the fluid pump.

31. The method of claim 30, wherein the power supply comprises a fuel cell and the step of activating the power supply comprises the step of providing a suitable reactant to a reaction chamber of the fuel cell to cause a chemical reaction.

32. The method of claim 31, wherein the step of providing a suitable reactant comprises the step of defeating a barrier separating the reactant from the reaction chamber within the fuel cell.

33. The method of claim 32, wherein the step of defeating a barrier comprises the step of removing a tear seal.

34. The method of claim 32, wherein the step of defeating a barrier comprises the step of breaking a frangible membrane.

35. The method of claim 32, wherein the barrier is selected from the group consisting of a frangible membrane, a tear seal, and any combination of the two.

36. The method of claim 30, wherein the step of operably connecting the power supply comprises inserting the power supply into the fluid pump.

37. The method of claim 31, wherein the fuel cell is a low temperature fuel cell.

38. The method of claim 30, wherein the tubing is a medical tubing and the power supply is integral to an outer surface of the medical tubing.

39. A method for delivering fluid through a lineset comprising the steps of:
providing an infusion system comprising a fluid pump and tubing having a first end in fluid communication with a fluid source and a second end in fluid communication with a delivery device;
providing a power supply affixed to a component of the infusion system other than the fluid pump;
operably connecting the power supply to the fluid pump;
activating the power supply to provide power to the fluid pump; and
pumping fluid through the tubing from the fluid source toward the second end of the tubing.

40. The method of claim 39, wherein the power supply comprises a fuel cell and the step of activating the power supply comprises the step of providing a suitable reactant to a reaction chamber of the fuel cell to cause a chemical reaction.

41. The method of claim 40, wherein the step of providing a suitable reactant comprises the step of removing a barrier separating the suitable reactant from the reaction chamber.

42. The method of claim 41, wherein the barrier is selected from the group consisting of a frangible membrane, a tear seal, and any combination of the two.

43. The method of claim 39, wherein the power supply is attached to the tubing.

44. The method of claim 43, wherein the power supply is integral to an outer surface of the tubing.

45. The method of claim 39, wherein the step of operably connecting the power supply comprises placing the fuel cell into the fluid pump.

46. The method of claim 40, wherein the fuel cell is a low temperature fuel cell.

47. A medical infusion system comprising a lineset having a means for attaching a first end to a reservoir and means for attaching a second end to another component, a means for engaging a durable pump to the lineset and means for controlling a fluid flow through the lineset, and a means for operatively connecting a means for supplying power, affixed to other than the durable pump, to the durable pump.

48. The medical infusion system of claim 47, wherein the means for supplying power is affixed to the lineset.

49. The medical infusion system of claim 48, wherein the lineset and means for supplying power are disposable.

50. The medical infusion system of claim 47, further comprising an auxiliary component attached to the lineset selected from the group consisting of a valve, a flow sensor, a pump, a pressure sensor, a feedback control input, a biological status sensor, other closed-loop type sensors, and any combination of such components.

51. The medical infusion system of claim 50, wherein the means for supplying power is affixed to the auxiliary component.

52. The medical infusion system of claim 1, wherein the means for supplying power comprises a fuel cell.

53. A medical lineset comprising:
tubing having a first end, a second end, and means for attaching each end to at least a first and second medical component;
means for supplying power attached to the tubing; and
means for activating the means for supplying power into operative connection with an electric component.

54. The medical lineset of claim 53, wherein the means for activating comprises a connector of the electric component.

55. The medical lineset of claim 53, wherein means for supplying power comprises a fuel cell.

56. The medical lineset of claim 55, wherein the fuel cell comprises a reactant source and a means for separating the reactant source from a reaction chamber.

57. The medical lineset of claim 56, wherein the means for separating is a barrier selected from the group consisting of a frangible membrane, a tear seal, and any combination of the two.

58. The medical lineset of claim 55, wherein the fuel cell is a low temperature fuel cell.
